(19) United States
(12) Patent Application Publication
(10) Pub. No.: US 2026/0041831 A1
(43) Pub. Date: Feb. 12, 2026

(54) METHOD AND APPARATUS

(71) Applicant: AWAK TECHNOLOGIES PTE LTD, Singapore (SG)

(72) Inventors: Suresha BELUR VENKATARAYA, Singapore (SG); Mandar Manohar GORI, Singapore (SG); Sanjay Kumar SINGH, Singapore (SG); Joel Preetham FERNANDES, Singapore (SG); Jason Tze Chern LIM, Singapore (SG); Marcin Bartlomiej PAWLAK, Singapore (SG); Sridhar CHIRUMARRY, Singapore (SG); Vinod Kumar GADI, Singapore (SG); Yue (Victoria) WANG, Singapore (SG); Bei Ming CHEN, Singapore (SG); Siti Noor Huda JAMALUDDIN, Singapore (SG); Abel Samson DSOUZA, Singapore (SG)

(73) Assignee: AWAK TECHNOLOGIES PTE LTD, Singapore (SG)

(21) Appl. No.: 19/104,282

(22) PCT Filed: Aug. 24, 2022
(86) PCT No.: PCT/SG2022/050604

§ 371 Date: Feb. 17, 2025

(51) Int. Cl. A61M 1/28

(52) U.S. Cl. CPC A61M 1/287; A61M 1/281; A61M 1/282; A61M 2202/0021; A61M 2202/04; A61M 2205/50; A61M 2210/1021

(57) ABSTRACT

Disclosed herein is a method of removing fluid from a subject, using the steps of (i) administering a first hypertonic solution comprising a sugar-based osmotic agent to a peritoneal cavity in the subject; (ii) allowing water from the subject to pass into the peritoneal cavity by osmosis, thereby forming a second hypertonic solution within the peritoneal cavity, the second hypertonic solution having a lower concentration of the sugar-based osmotic agent than the first hypertonic solution; (iii) withdrawing a portion of the second hypertonic solution from the peritoneal cavity and combining the withdrawn second hypertonic solution with a sugar concentrate to form a third hypertonic solution; (iv) administering the third hypertonic solution to the peritoneal cavity to form a fourth hypertonic solution within the peritoneal cavity by mixing of the second and third hypertonic solutions; and (v) repeating steps (ii) to (iv) for a desired treatment time. Also disclosed herein is an apparatus suitable for use with the method.

### FIELD OF INVENTION

The current invention relates to a method and apparatus useful for reducing the fluid in a subject in need of fluid reduction who has acute or chronic kidney disease.

### BACKGROUND

The listing or discussion of a prior-published document in this specification should not necessarily be taken as an acknowledgement that the document is part of the state of the art or is common general knowledge.

In CKD (chronic kidney disease) patients, the kidneys are unable to remove excess fluid & toxins from the patient. In the case of insufficient fluid removal from the patient, this will eventually lead to fluid retention which can cause multiple health complications such as high blood pressure, heart problems and more. Dialysis aims to remove both toxins and fluid from the patient.

The current standard of care APD/CAPD modalities introduce a hypertonic solution with each Fill to ensure that sufficient osmotic pressure is present to generate ultrafiltration (UF). This solution is left to dwell within the patient for a fixed duration. During this dwell phase, the solution is diluted due to the ultrafiltration that is generated from the patient, as well as lymphatic absorption of glucose by the patient. The resultant fluid at the end of each cycle is drained out and another Fill of hypertonic solution is then reintroduced to the patient in the next cycle. This process is repeated until the prescribed number of cycles are completed. FIG. 1 illustrates how dialysate tonicity drops over time which each cycle and how multiple instances of hypertonic solution is introduced to the patient.

Therefore, there exists a need for a system that maintains a steady and mildly hypertonic solution which allows consistent UF generation throughout the entire therapy.

### SUMMARY OF INVENTION

Aspects and embodiments of the invention will now be described by reference to the following numbered embodiments.

1. A method of removing fluid from a subject, comprising the steps of:
(i) administering from 200 mL to a tolerable maximum volume for the subject of a first hypertonic solution comprising a sugar-based osmotic agent to a peritoneal cavity in the subject;
(ii) allowing water from the subject to pass into the peritoneal cavity by osmosis, thereby forming a second hypertonic solution within the peritoneal cavity, the second hypertonic solution having a lower concentration of the sugar-based osmotic agent than the first hypertonic solution;
(iii) withdrawing from 100 to 500 mL of the second hypertonic solution from the peritoneal cavity and combining the withdrawn second hypertonic solution with from 0.3 to 6.8 mL of a sugar concentrate to form a third hypertonic solution, the sugar concentrate having a concentration of from 0.25 to 0.9 g/mL;
(iv) administering the third hypertonic solution to the peritoneal cavity to form a fourth hypertonic solution within the peritoneal cavity by mixing of the second and third hypertonic solutions; and
(v) repeating steps (ii) to (iv) every 5 to 30 minutes for a desired treatment time.

2. The method according to Clause 1, wherein step (i) comprises administering from 300 to 4,000 mL, such as from 400 to 3,000 mL such as from 500 to 2,500 mL of a first hypertonic solution comprising a sugar-based osmotic agent to the peritoneal cavity.

3. The method according to Clause 1 or 2, wherein step (i) comprises administering from 1,000 to 2,500 mL of a first hypertonic solution comprising a sugar-based osmotic agent to the, peritoneal cavity.

4. The method according to any one of the preceding clauses, wherein step (iii) comprises withdrawing from 200 to 400 mL of the second hypertonic solution, optionally from 250 to 300 mL.

5. The method according to any one of the preceding clauses, wherein step (iii) comprises combining the withdrawn second hypertonic solution with from 0.3 to 6.8 mL of a sugar concentrate, optionally from 0.33 to 3.0 mL, such as from 0.4 to 2.8 mL.

6. The method according to Clause 5, wherein step (iii) comprises combining the withdrawn second hypertonic solution with from 0.5 to 2.4 mL, such as from 0.6 to 2.0 mL of a sugar concentrate, optionally wherein step (iii) comprises combining the withdrawn second hypertonic solution with a sugar concentrate having a concentration of from 0.65 to 0.85 g/mL, such as about 0.7 g/mL.

7. The method according to any one of the preceding clauses, wherein step (iii) further comprises the periodical step of removing fluid from the subject by:
(a) withdrawing a volume of the second hypertonic solution that comprises a desired second hypertonic volume and an excess second hypertonic volume, and removing the excess second hypertonic volume before forming the third hypertonic solution; or
(b) withdrawing a volume of the second hypertonic solution that comprises a desired second hypertonic volume and an excess second hypertonic volume, then forming a third hypertonic solution that comprises a desired third hypertonic volume and an excess third hypertonic volume and removing the excess third hypertonic volume before administering the third hypertonic solution to the subject.

8. The method according to any one of the preceding clauses, wherein step (v) comprises repeating steps (ii) to (iv) every 7 to 17 minutes for a desired treatment time, optionally every 7.5 to 15 minutes, such as about every 7.5 minutes or about every 15 minutes.

9. The method according to any one of the preceding clauses, wherein the desired treatment time is from 7 to 10 hours.

10. The method according to any one of the preceding clauses, wherein step (iii) comprises passing the withdrawn second hypertonic solution through a dialysis sorbent; or passing the third hypertonic solution through a dialysis sorbent.

11. The method according to Clause 10, further comprising an initial step of saturating the sorbent with the sugar concentrate.

12. The method according to any one of the preceding clauses, wherein the sugar concentrate comprises a sugar-based osmotic agent, optionally wherein the sugar based osmotic agent is selected from one or both of glucose and icodextrin, optionally wherein the sugar concentrate comprises glucose.

13. The method according to any one of the preceding clauses, wherein the sugar-based osmotic agent concentration of the withdrawn second hypertonic solution in each repetition of step (ii) varies by less than 50%, such as less than 40%, relative to the initial sugar-based osmotic agent concentration of the withdrawn second hypertonic solution the first time step (ii) is performed.

14. The method according to any one of the preceding clauses, wherein the third hypertonic solution has a higher sugar-based osmotic agent concentration than the first hypertonic solution.

15. The method according to any one of the preceding clauses, wherein the fourth hypertonic solution has a sugar-based osmotic agent concentration that is less than or equal to the sugar-based osmotic agent concentration of the first hypertonic solution.

16. An apparatus comprising:
a first pump fluidly connectable to a subject's peritoneum;
a sugar concentrate supply pump connectable to a source of sugar concentrate;
a storage chamber; and
a first fluid flow path from the first pump to the storage chamber;
where the first pump is configured to pump fluid in either direction along the first fluid flow paths,
such that when in use:
   fluid can be drawn from a subject's peritoneum along the first fluid flow path by the first pump to the storage chamber; and
   the sugar concentrate supply pump is configured to supply a sugar concentrate to fluid flowing along the first fluid flow path.

17. The apparatus according to Clause 16, wherein the apparatus is configured to be connected to a controller configured to operate the apparatus, optionally wherein the controller is configured to implement a method according to any one of Clauses 1 to 15.

18. The apparatus according Clause 16 or Clause 17, further comprising one or more mixers located on the first fluid flow path.

19. The apparatus according to any one of Clauses 16 to 20, wherein the apparatus further comprises:
a dialysis sorbent situated in the first fluid flow path; and
a second fluid flow path from the first pump to the storage chamber that bypasses the sorbent, where the controller can select whether to use the first or the second fluid flow path for any fluid flow operation, such that when in use:
   fluid can be drawn from a subject's peritoneum along the first fluid or second flow path by the first pump to the storage chamber; and
the sugar concentrate supply pump is configured to supply a sugar concentrate to fluid flowing along the first fluid or second flow paths.

20. The apparatus according to Clause 19, further comprising one or more valves configured to selectively enable fluid flow through one of the first and second fluid flow paths.

21. The apparatus according to Clause 19 or 20, further comprising one or more mixers located on the first and second fluid flow paths.

22. The apparatus according to any one of Clauses 19 to 21, wherein the sugar concentrate supply pump is configured to supply sugar concentrate to fluid in the second fluid flow path.

23. The apparatus according to any one of Clauses 19 to 22, wherein the dialysis sorbent is situated upstream or downstream of the storage chamber in the first fluid flow path.

### DRAWINGS

FIG. 1 depicts an example of changes in dialysate tonicity which each cycle of a standard APD/CAPD therapy.

FIG. 2A to FIG. 2B depicts one embodiment of the disclosed glucose management system in peritoneal dialysis.

FIG. 3A to FIG. 3C depicts one embodiment of the disclosed glucose management system in peritoneal dialysis.

FIG. 4A to FIG. 4B depicts one embodiment of the disclosed glucose management system in peritoneal dialysis.

FIG. 5A to FIG. 5C depicts one embodiment of the disclosed glucose management system in peritoneal dialysis with a 3-way valve with glucose dosing in tidal outflow and in UF only outflow.

FIG. 6A to FIG. 6C depicts one embodiment of the disclosed glucose management system in peritoneal dialysis with a 3-way valve with glucose dosing in tidal inflow and in UF only inflow.

FIG. 7A to FIG. 7C depicts one embodiment of the disclosed glucose management system in peritoneal dialysis with two 2-way valves with glucose dosing in tidal outflow, and in UF only outflow.

FIG. 8A to FIG. 8C depicts one embodiment of the disclosed glucose management system in peritoneal dialysis with two 2-way valves with glucose dosing in tidal inflow and in UF only inflow.

FIG. 9A and FIG. 9B depicts one embodiment of the disclosed glucose management system in peritoneal dialysis with outflow glucose dosing and with no sorbent configuration.

FIG. 10A and FIG. 10B depicts one embodiment of the disclosed glucose management system in peritoneal dialysis with inflow glucose dosing and with no sorbent configuration.

FIG. 11 depicts the outflow glucose concentration at Setting 1, Setting 2 & Setting 3.

FIG. 12 depicts the UF generated per gram of glucose absorbed (n=number of days on a modality).

FIG. 13 depicts the UF generated per gram of glucose exposure (n=number of days on a modality; * p-value<0.05).

FIG. 14 depicts the glucose tonicity changes with application of AGMS (dotted lines) vs standard APD therapies (lines).

FIG. 15 depicts an Initial Fill dialysate setting and glucose dose setting for an apparatus and method according to embodiments of the current invention.

FIG. 16A to FIG. 16D depict an apparatus and flow scheme from an apparatus used in Example 1 below. These figures relate to the outflow (FIG. 16A) and inflow (FIG. 16B) cycles that pass through a dialysis sorbent as well as to outflow (FIG. 16A) and inflow (FIG. 16B) cycles that do not pass through a dialysis sorbent.

### DESCRIPTION

It has been surprisingly discovered that some or all of the problems associated problems associated with the current methods of care can be solved through the use of a tidal volume method and apparatus. Thus, disclosed herein is an apparatus that uses a tidal therapy that differs from the regular APD/CAPD modalities by continuously regenerating a small volume of dialysate (relative to the Initial Fill volume). This is known as the Tidal Volume. At each cycle, the apparatus can introduce a small volume of glucose to ‘top up’ and counter any loss of osmotic pressure due to the dilution effect of UF generated as well as any lymphatic absorption of glucose by the patient. The aim of this apparatus and method is to maintain a steady and mildly hypertonic solution which allows consistent UF generation throughout the entire therapy.

The AWAK Advanced Glucose Management System (AWAK AGMS) discussed herein was designed to allow a physician to adjust the amount of glucose that is dosed during each tidal cycle in order to regulate and target the required UF removal.

Thus, in a first aspect of the invention, there is provided an apparatus comprising:
a first pump fluidly connectable to a subject's peritoneum;
a sugar concentrate supply pump connectable to a source of sugar concentrate;
a storage chamber; and
a first fluid flow path from the first pump to the storage chamber;
where the first pump is configured to pump fluid in either direction along the first fluid flow paths,
such that when in use:
   fluid can be drawn from a subject's peritoneum along the first fluid flow path by the first pump to the storage chamber
   and
   the sugar concentrate supply pump is configured to supply a sugar concentrate to fluid flowing along the first fluid flow path.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

The terms “subject” and “subjects” include references to mammalian (e.g. human) subjects. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

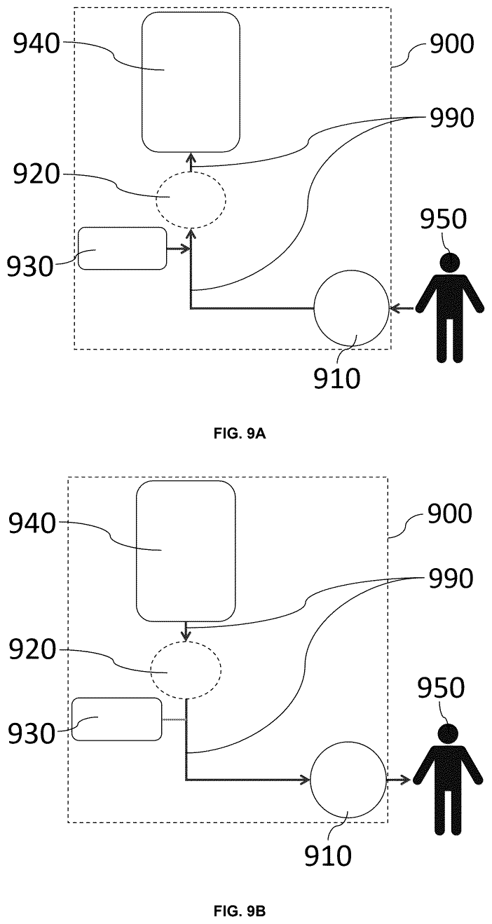

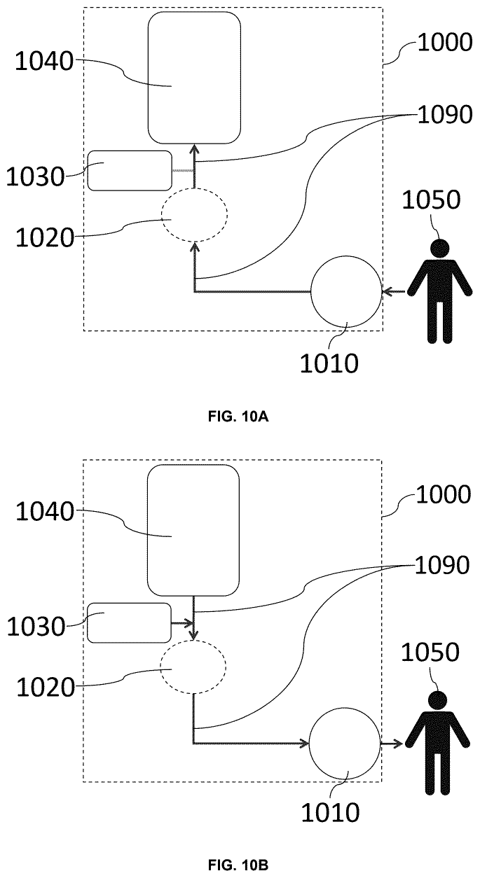

The apparatus will be briefly discussed by reference to FIG. 9. As noted above the apparatus 900 comprises
a first pump 910 fluidly connectable to a subject's 950 peritoneum;
a sugar concentrate supply pump 930 connectable to a source of sugar concentrate;
a storage chamber 940; and
a first fluid flow path 990 from the first pump 910 to the storage chamber 940;
where the first pump 910 is configured to pump fluid in either direction along the first fluid flow path,
such that when in use:
   fluid can be drawn from a subject's peritoneum along the first fluid flow path by the first pump to the storage chamber; and
   the sugar concentrate supply pump is configured to supply a sugar concentrate to fluid flowing along the first fluid flow path.

While not shown, the apparatus of FIG. 9 may be connected to a controller that configured to operate the apparatus, where the controller can select whether to use the first or the second fluid flow path for any fluid flow operation. It will be appreciated that any suitable controller may be used. For example, the controller may be an Arduino Due and relevant supporting electronics, which may include pressure sensors etc.

It will be appreciated that the sugar concentrate can be added either in an outflow sense (i.e. when fluid is being drawn from a subject), in an inflow sense (i.e. when fluid is being returned to a subject) or in both senses, depending on the need of the subject and the physician's instructions.

It will be appreciated that there will be a certain amount of turbulence associated with the flow of the fluid resulting in mixing of the sugar concentrate with the bulk of the fluid through the movement of the fluid through the system, storage in the storage tank or while in the peritoneal cavity of a subject. As such there may be no need to include any mixing means or apparatus as part of the apparatus. However, in some embodiments of the invention, a mixing means or apparatus, such as a mixer 920 may be placed within the first fluid flow path. As will be appreciated, the exact location of the mixer(s) will depend on how the apparatus is intended to function. For example, the mixer 920 may be a single mixer that is placed downstream relative to the direction of flow used to introduce the sugar concentrate. As shown in FIG. 9A, the mixer 920 is placed between the sugar concentrate supply pump 930 and the storage chamber 940. In other embodiments, the mixer 920 may be placed so that it is upstream of the sugar concentrate supply pump 930 in an inflow sense, such that the sugar concentrate supply pump 930 and the storage chamber 940 are not separated by the mixer. This allows for the introduction and mixing of the sugar concentrate during an inflow phase of the apparatus. Alternatively, there may be more than one mixer in the first fluid flow path, allowing mixing to take place both in an inflow and outflow phase of the apparatus. It will be understood that the placement of mixers discussed here is general and applies to all other embodiments discussed herein.

As will be appreciated, the apparatus disclosed herein may be used in conjunction with a control means or apparatus. This control means or apparatus is configured to operate the apparatus and may be, in particular embodiments mentioned herein, configured to implement the methods described hereinbelow. As will be appreciated, the control means or apparatus may be a reusable component that can be connected to and then removed from a disposable apparatus.

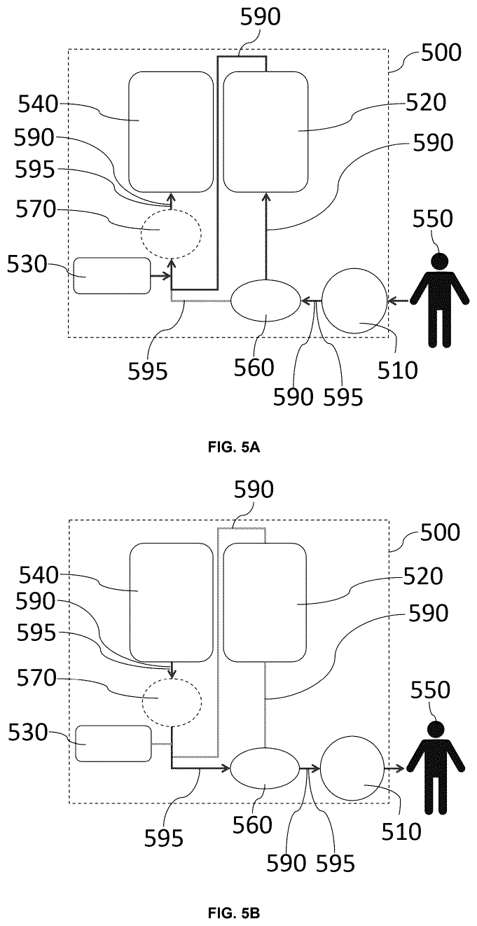

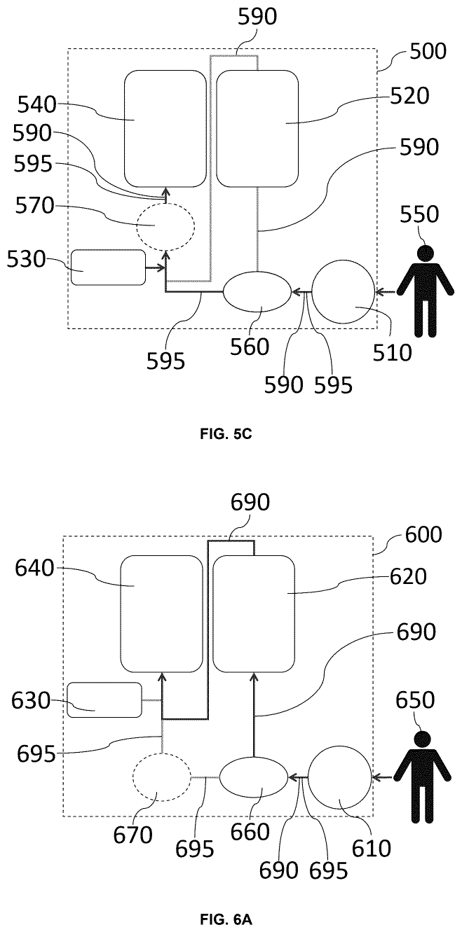

The above apparatus may be suitable for the removal of fluid from a subject only, without necessarily dealing with the removal of a substantial amount of toxins from the subject. With that in mind, further embodiments of the apparatus may introduce a sorbent to the apparatus that enables toxins to be removed, thereby allowing peritoneal dialysis to take place at the same time as removing fluid from the subject. Such an apparatus will be briefly discussed by reference to FIG. 5. As noted above the apparatus 500 comprises
a first pump 510 fluidly connectable to a subject's 550 peritoneum;
a sugar concentrate supply pump 530 connectable to a source of sugar concentrate;
a storage chamber 540;
a first fluid flow path 590 from the first pump 510 to the storage chamber 540;
a dialysis sorbent 520 situated in the first fluid flow path; and
a second fluid flow 595 path from the first pump 510 to the storage chamber 540 that bypasses the sorbent 512;
where the first pump 510 is configured to pump fluid in either direction along the first and second fluid flow paths,
such that when in use:
   fluid can be drawn from a subject's peritoneum along the first fluid or second flow path by the first pump to the storage chamber; and
   the sugar concentrate supply pump is configured to supply a sugar concentrate to fluid flowing along the first fluid or second flow paths.

While not shown, the apparatus of FIG. 5 may be connected to a controller that configured to operate the apparatus, where the controller can select whether to use the first or the second fluid flow path for any fluid flow operation.

The dialysis sorbent mentioned herein may be any suitable dialysis sorbent and is not particularly limited. The only requirement is that it can be packed into a suitable chamber within the apparatus. Examples of sorbents include, but are not limited to those described in PCT Application No. PCT/SG2009/000229, which is hereby incorporated by reference.

As will be appreciated, the dialysis sorbent will be housed in a suitable chamber within the apparatus disclosed herein. If the apparatus is disposable, the sorbent may be housed within the chamber directly. However, if the apparatus is intended to be partly re-usable, the sorbent may be stored within a separate sorbent cartridge that may be placed into the apparatus before use. The former arrangement, where the sorbent is directly held within a chamber, thereby allowing the apparatus to be disposable in nature after a single use, which may be beneficial for hygiene reasons.

As will be appreciated, the pumps may be part of a permanent apparatus section and are not disposable. They may be connected to the controller for this purpose. The apparatus may have a disposable section which consists of the tubing, flow paths, dialysis sorbent (when present) and storage chamber, plus any connections to, for example, an ultrafiltration bag.

The apparatus of FIG. 5 allows one to select at each outflow phase whether to pass the fluid from a subject through the sorbent or not. This may be achieved by the use of any suitable means or apparatus that allows for such control. For example the apparatus may make use of one or more values configured to selectively enable fluid flow through one of the first and second fluid flow paths. The embodiment of FIG. 5 may include a three-way valve 560 that enables the selection of the desired flow path under the influence of the controller.

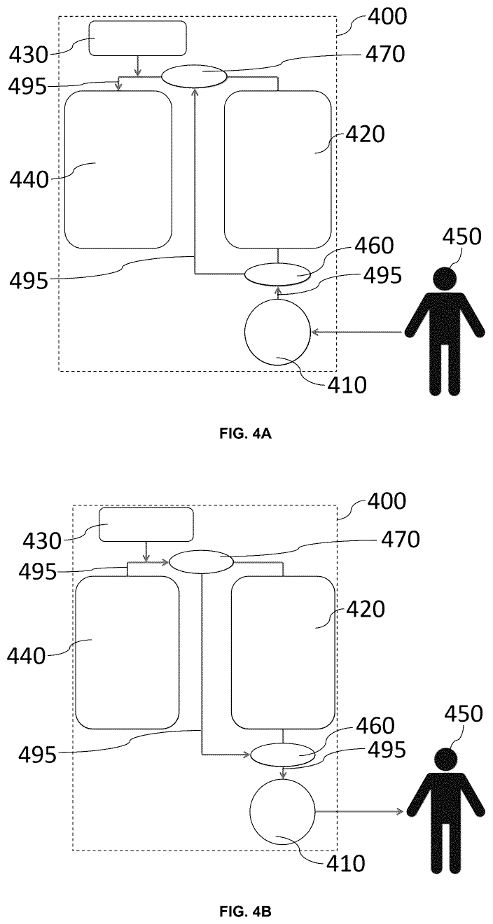

An alternative embodiment of the apparatus may make use of two or more valves configured to selectively enable fluid flow through one of the first and second fluid flow paths. For example, the embodiment depicted in FIG. 4 makes use of two values 460 and 470, which allows for greater control. That is, the use of two valves in the configuration depicted allows for the sorbent to be used either during the inflow phase or the outflow phase, as desired. In other words, in embodiments of the invention that may be mentioned herein, the dialysis sorbent may be situated upstream or downstream of the storage chamber in the first fluid flow path.

As will be appreciated, one or more mixers may be located in the first and second flow paths (e.g. 570 in FIG. 5) and these may be configured in a similar manner to that described above.

In embodiments that may be mentioned herein, the sugar concentrate supply pump may be configured to supply sugar concentrate to fluid in the second fluid flow path. This is shown in FIG. 5. For the avoidance of doubt, the sugar concentrate supply pump may be configured to supply sugar concentrate to fluid in the first fluid flow path should that be desired.

Further embodiments of the apparatus will be discussed by reference to the method for which the apparatus disclosed above in general terms is useful for. Thus, in a second aspect of the invention, there is disclosed a method of removing fluid from a subject, comprising the steps of:
(i) administering from 200 mL to a tolerable maximum volume for the subject of a first hypertonic solution comprising a sugar-based osmotic agent to a peritoneal cavity in the subject;
(ii) allowing water from the subject to pass into the peritoneal cavity by osmosis, thereby forming a second hypertonic solution within the peritoneal cavity, the second hypertonic solution having a lower concentration of the sugar-based osmotic agent than the first hypertonic solution;
(iii) withdrawing from 100 to 500 mL of the second hypertonic solution from the peritoneal cavity and combining the withdrawn second hypertonic solution with from 0.3 to 6.8 mL of a sugar concentrate to form a third hypertonic solution, the sugar concentrate having a concentration of from 0.25 to 0.9 g/mL;
(iv) administering the third hypertonic solution to the peritoneal cavity to form a fourth hypertonic solution within the peritoneal cavity by mixing of the second and third hypertonic solutions; and
(v) repeating steps (ii) to (iv) every 5 to 30 minutes for a desired treatment time.

When used herein, the term “tolerable maximum volume” is subjecting and will be determined by each subject who undergoes the method of treatment. Examples of the maximum tolerable volume may include volumes up to, and exceeding 4,000 mL. Examples of suitable volumes that may be used include, but are not limited to from 300 to 4,000 mL, such as from 400 to 3,000 mL such as from 500 to 2,500 mL of a first hypertonic solution comprising a sugar-based osmotic agent to the peritoneal cavity.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in step (i) of the method, the following volume ranges of the first hypertonic solution are expressly contemplated:
from 200 to 300 mL, from 200 to 400 mL, from 200 to 500 mL, from 200 to 1,000 mL, from 200 to 2,500 mL, from 200 to 3,000 mL, from 200 to 4,000 mL, from 200 mL to a tolerable maximum volume for the subject;
from 300 to 400 mL, from 300 to 500 mL, from 300 to 1,000 mL, from 300 to 2,500 mL, from 300 to 3,000 mL, from 300 to 4,000 mL, from 300 mL to a tolerable maximum volume for the subject;
from 400 to 500 mL, from 400 to 1,000 mL, from 400 to 2,500 mL, from 400 to 3,000 mL, from 400 to 4,000 mL, from 400 mL to a tolerable maximum volume for the subject;
from 500 to 1,000 mL, from 500 to 2,500 mL, from 500 to 3,000 mL, from 500 to 4,000 mL, from 500 mL to a tolerable maximum volume for the subject;
from 1,000 to 2,500 mL, from 1,000 to 3,000 mL, from 1,000 to 4,000 mL, from 1,000 mL to a tolerable maximum volume for the subject;
from 2,500 to 3,000 mL, from 2,500 to 4,000 mL, from 2,500 mL to a tolerable maximum volume for the subject;
from 3,000 to 4,000 mL, from 3,000 mL to a tolerable maximum volume for the subject; and
from 4,000 mL to a tolerable maximum volume for the subject.

The above analysis may be applied to all other sets of numerical ranges disclosed herein.

In the method disclosed herein, any suitable amount between 100 to 500 mL of the second hypertonic solution may be withdrawn from the peritoneal cavity. For example, the step (iii) may comprise withdrawing from 200 to 400 mL of the second hypertonic solution, optionally from 250 to 300 mL.

In step (iii) the withdrawn volume of the second hypertonic solution is combined with a sugar concentrate solution to form a third hypertonic solution. The amount of the sugar concentrate solution may have a concentration of from 0.25 to 0.9 g/mL of the sugar-based osmotic agent (or a combined total concentration in this range if there is more than one sugar-based osmotic agent present). For example, the concentration of the sugar concentrate may be from 0.65 to 0.85 g/mL, such as about 0.7 g/mL. The amount of the sugar concentrate that is combined with the withdrawn second hypertonic solution is from 0.3 to 6.8 mL. For example, the amount of the sugar concentrate that is combined with the withdrawn second hypertonic solution may be 0.3 to 6.8 mL of a sugar concentrate, such as from 0.33 to 3.0 mL, such as from 0.4 to 2.8 mL, such as from 0.5 to 2.4 mL, such as from 0.6 to 2.0 mL.

As will be appreciated, the method disclosed herein is a tidal system. As such, the steps of the method disclosed above are repeated in a cyclical manner over a suitable period of time to have the desired effect. As such, steps (ii) to (iv) of the method may be repeated every 5 to 30 minutes (e.g. every 10 to 20 minutes) for a desired treatment time. For example, steps (ii) to (iv) may be repeated every 7 to 17 minutes, such as every 7.5 to 15 minutes, such as about every 7.5 minutes or about every 15 minutes for a desired treatment time.

Any suitable period of time determined by the skilled physician may be used herein. Examples of suitable desired treatment times may be from 7 to 10 hours, though longer (or shorter) times may be selected by the physician based upon their own knowledge of the subject in question.

In embodiments of the invention, the method may make use of a dialysis sorbent (which may be placed into an apparatus as discussed briefly above). As such, step (iii) may further comprise passing the withdrawn second hypertonic solution through a dialysis sorbent; or passing the third hypertonic solution through a dialysis sorbent. In certain embodiments, the sorbent may be subjected to an initial step of being saturated with the sugar concentrate before it is used. This step (if used) is intended to speed up the saturation of the sorbent with the sugar-based osmotic agent present in the sugar concentrate. However, this step is not necessary, as the dialysate coming from the patient already includes the sugar-based osmotic agent (and glucose etc. from the subject). As such, the saturation of the sorbent with these sugar-based substances will happen even without this pre-saturation step, albeit potentially over a longer period of time.

As noted herein, the method makes use of a sugar concentrate. This sugar concentrate may be any suitable sugar based osmotic agent. For example, the sugar based osmotic agent may be selected from one or both of glucose and icodextrin. In particular embodiments of the invention the sugar concentrate may comprise (or be) glucose. As will be appreciated, the same sugar-based osmotic agents may be used in the first hypertonic solution.

The maximum total dosage of the sugar based osmotic agent per treatment may be based on the current gold-standard of care, which is 14 L of an aqueous 2.5 wt % glucose solution. It will be appreciated that the methods disclosed herein enables the amount of glucose (or other sugar based osmotic agents) to be reduced significantly compared to this maximum value.

In embodiments of the invention that may be mentioned herein, the sugar-based osmotic agent concentration of the withdrawn second hypertonic solution in each repetition of step (ii) varies by less than 50%, such as less than 40%, relative to the initial sugar-based osmotic agent concentration of the withdrawn second hypertonic solution the first time step (ii) is performed.

In embodiments of the invention, the third hypertonic solution may have a higher sugar-based osmotic agent concentration than the first hypertonic solution.

In embodiments of the invention, the fourth hypertonic solution may have a sugar-based osmotic agent concentration that is less than or equal to the sugar-based osmotic agent concentration of the first hypertonic solution.

A possible operational embodiment of the apparatus and method will now be provided from the perspective of a user and as depicted in FIG. 15. In this embodiment, the method makes use of a set of sugar concentrates and enables the user to select the concentrate for the initial fill and for the subsequent sugar dose settings during the run of the method. For example, in this embodiment of the method disclosed herein the user can elect to use one of many possible dialysate concentrations (e.g. 1.5 wt % Dextrose, 2.3 wt % Dextrose, 2.5 wt % Dextrose, 4.25 wt % Dextrose, and Icodextrin 7.5 wt %) for the Initial Fill. The physician/user then configures the device accordingly for the Initial Fill dialysate type. Once the dialysate type has been configured, the physician/user will be given a choice of up to six sugar-based osmotic agent dose settings (1, 2, 3, 4, 5 and 6) to allow better control over the UF generation for different patient profiles (FIG. 15). These settings will dispense 0.4 mL, 0.8 mL, 1.2 mL, 1.6 mL, 2.0 mL and 2.4 mL of a 70 wt % glucose concentrate, respectively.

As not all patients have the same transport characteristics, some adjustment and setting titration may need to be made by the physician to ensure that the right amount of glucose (or other sugar-based osmotic agent) is dosed so as to ensure sufficient UF is generated.

The physician will be provided with a prescription guide for reference, but it will be up to the discretion of the physician to prescribe an appropriate Initial Fill dialysate and glucose setting combination according to the need of the individual subjects under their care. This is because there may be more than one configuration that can give the same ultrafiltration for the same patient (for example, Dextrose 1.5 wt % with Setting 4 and Dextrose 2.5 wt % with Setting 2). In cases like these, the physician may choose to select one prescription over the other due to various reasons (e.g. to maintain the patient on the same dialysate type as their standard of care so as to reduce the burden of maintaining different dialysate types, or the physician may choose to select a combination that maximizes ultrafiltration efficiency.

These settings may also change on a daily basis based on the requirement of the patient on that day. For example, if the patient is on a prescription requiring an Initial Fill of 1.5 wt % and Setting 3, the patient may choose to increase the glucose dosing to Setting 4 if they see signs of oedema (fluid retention) or reduce the glucose dosing to Setting 2 if they see signs of dehydration. The combination of Initial Fill tonicity and glucose setting may also change over time according to any changes in patient's membrane transport characteristics.

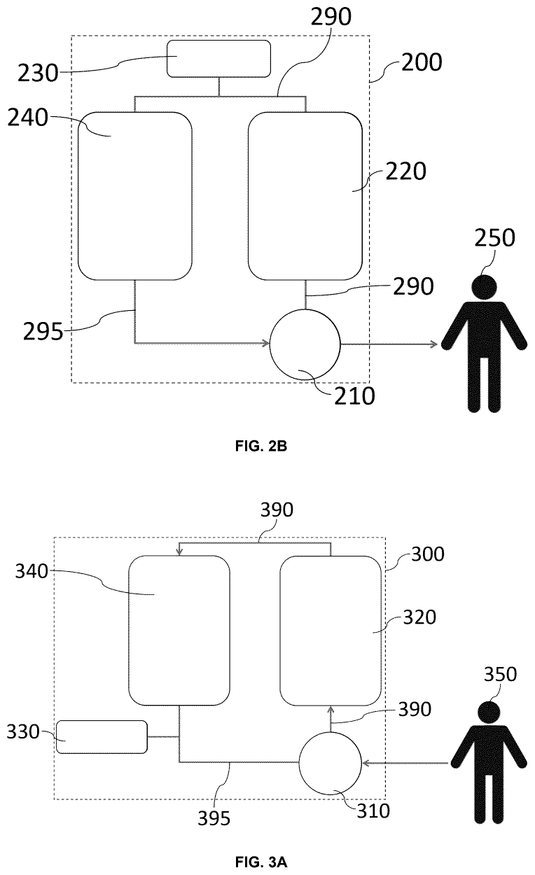

We will now describe the operation of the apparatus and method in some more detail by reference to FIGS. 2 to 10.

In some representative or exemplary embodiments of the present disclosure, with reference to FIG. 2 (FIGS. 2A and 2B), there is disclosed an apparatus 200 and a subject 250. The apparatus 200 includes a first pump 210, a dialysis sorbent 220, a sugar concentrate supply pump 230, and a storage chamber 240. There is a first fluid flow path 290 and a second fluid flow path 295. The first fluid flow path 290 passes through the first pump 210, through the dialysis sorbent 220 to the storage chamber 240. The second fluid flow path 295 passes from the first pump 210 to the storage chamber 240 directly without passing through the dialysis sorbent.

FIG. 2A depicts the use of the apparatus 200 in an outflow phase (i.e. fluid is drawn from the subject into the storage chamber). This may represent the first running of step (ii) after the loading of the subject with the first hypertonic solution to the peritoneal cavity of a subject or a subsequent running of step (ii). In the configuration depicted, the first pump 210 draws a portion of the second hypertonic solution into the apparatus and directs it through the sorbent 220, after passing through the sorbent, the sugar concentrate supply pump 230 (which is connected to a sugar concentrate as described above) provides a dose of said concentrate to the second hypertonic solution to form the third hypertonic solution, which then enters the storage chamber 240.

FIG. 2B depicts the use of the apparatus 200 in an inflow phase (i.e. fluid is returned to the subject from the storage chamber). In the configuration depicted, the first pump 210 draws the third hypertonic solution from the storage chamber 240 through the first pump 210 and back into the subject 250. As such, the third hypertonic solution does not pass through the sorbent on its return to the subject.

As will be appreciated, there is a controller system that controls the operation of the apparatus during the method through the outflow and inflow operations, and also controls the dosage of the sugar concentrate. The sugar concentrate may be supplied as one or more boluses or as a continuous stream during the outflow phase.

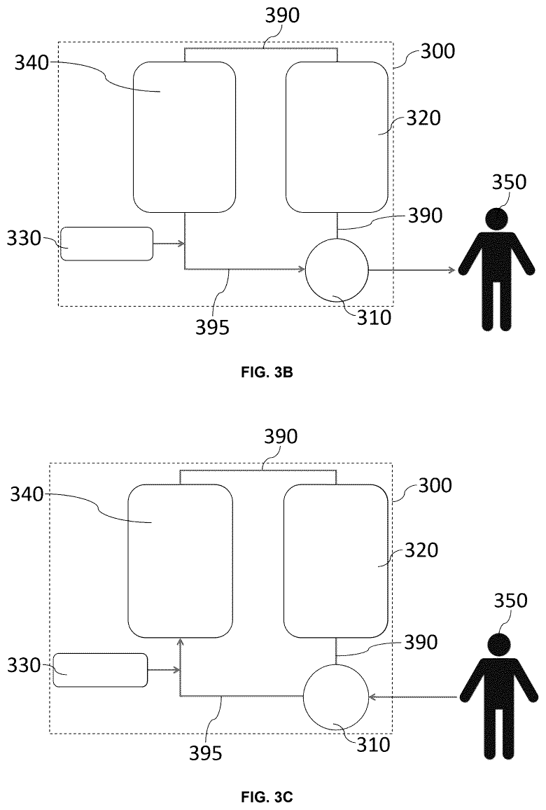

FIG. 3 (FIGS. 3A and 3B), depicts an apparatus 300 and a subject 350. The apparatus 300 includes a first pump 310, a dialysis sorbent 320, a sugar concentrate supply pump 330, and a storage chamber 340. There is a first fluid flow path 390 and a second fluid flow path 395. The first fluid flow path 390 passes through the first pump 310, through the dialysis sorbent 320 to the storage chamber 340. The second fluid flow path 395 passes from the first pump 310 to the storage chamber 340 directly without passing through the dialysis sorbent.

The apparatus of FIGS. 3A and 3B are essentially identical to the embodiment depicted in FIGS. 2A and 2B, but for the positioning of the sugar concentrate supply pump 330, which is located downstream of the storage chamber, rather than being upstream of it in FIGS. 2A and 2B. As such the apparatus and method operate in much the same manner as that depicted in FIGS. 2A and 2B in the outflow and inflow operations, but for the fact that the sugar concentrate is now supplied in the inflow phase (see FIG. 3B).

In the arrangements of FIGS. 2 and 3, the sugar-based osmotic agent is added to the dialysate after it has been regenerated by a sorbent. This helps to prevent the sorbent from absorbing the sugar-based osmotic agent that has been added to the third hypertonic solution. In both cases, a key consideration is to ensure that the glucose is mixed/diluted into the Tidal Volume of the dialysate before it passed back to the subject's peritoneal cavity. This is to prevent the sugar concentrate (e.g. 70 wt % glucose) from coming into direct contact with the subject's peritoneal membrane.

In another embodiment, the advanced glucose management system (AGMS) can also be operated in a ‘UF only’ mode which allows additional UF to be removed from the patient without any sorbent clearance of toxin occurring. Thus, in the ‘UF only’ mode, the dialysis sorbent is bypassed.

In this case, the outflow arrangement depicted in FIG. 3A is replaced by the arrangement of FIG. 3C, where the second hypertonic solution passes through only the second fluid flow path and returns to the subject through the same flow path in the inflow direction (see FIG. 3B). In addition, the sugar concentrate pump may supply the sugar concentrate in one or both of the outflow and inflow phases when the apparatus is operated in UF mode. As will be appreciated, in this UF mode, it may be possible to reduce the amount of the sugar concentrate introduced into the hypertonic solution in both the outflow and inflow directions, which may allow for a greater dilution/mixing of the sugar concentrate before the hypertonic solution is introduced back into the subject.

As will be appreciated, the controller/pump configuration or suitable flow control means or apparatus may enable the bypass of the sorbent as depicted in FIG. 3C.

The AWAK AGMS can also be also be applied for UF removal for heart patients who do not require toxin clearance. Water removal can help to improve blood pressure and maintain normotension. The system to achieve this is similar to that shown in FIGS. 3B and 3C, except without the sorbent installed. Alternatively, the sorbent (and its chambers) can be completely removed, as depicted by FIGS. 9A and 9B, which operate in the manner depicted for FIGS. 3C and 3B, respectively, but for the introduction of a mixer 920.

FIG. 4 depicts another embodiment of how UF mode can be achieved to bypass the sorbent with outflow and inflow. In FIG. 4, there is an apparatus 400 and a subject 450. The apparatus 400 includes a first pump 410, a dialysis sorbent 420, a sugar concentrate supply pump 430, a storage chamber 440, a first valve 460, a second valve 470, a first fluid flow path (not shown) and a second fluid flow path 495.

The sugar concentrate supply pump 430 is supplied to the fluid at a location between the storage chamber 440 and the second valve 470.

With reference to FIG. 4A, during the outflow stage in ‘UF only’ mode, the fluid flows from the subject's 450 peritoneum along the second fluid flow path through the first pump 410, the first valve 460, the second valve 470 and to the storage chamber 440. The sugar concentrate supply pump 430 supplies a sugar concentrate to the fluid flowing along the second fluid flow path. With reference to FIG. 4B, during the inflow stage in ‘UF only’ mode, the fluid flows from the storage chamber 440 along the second fluid flow path through the second valve 470, the first valve 460, the first pump 410 and to the subject's 450 peritoneum. As will be appreciated, a sugar concentrate may be supplied to the fluid flowing along the second fluid flow path during the outflow stage, the inflow stage, or both the outflow and inflow stages in ‘UF only’ mode.

As will be clear, when operating in toxin removal mode in the outflow phase, the second hypertonic solution passes through the first pump 410, the first valve 460, the sorbent 420, the second valve 470 and to the storage chamber 440. The inflow phase is identical to that described above for FIG. 4B in this toxin removal mode.

FIG. 5 depicts an apparatus 500 and a subject 550. The apparatus 500 includes a first pump 510, a dialysis sorbent 520, a sugar concentrate supply pump 530, a storage chamber 540, a 3-way valve 560, and an optional mixer 570 supplied between the storage chamber 540 and the sugar concentrate supply pump 530, a first fluid flow path 590 and a second fluid flow path 595.

The sugar concentrate supply pump 530 can supply the sugar concentrate to the fluid at a location between the storage chamber 540 and the 3-way valve 560 (via the second fluid flow path in an inflow or outflow direction) and between the storage chamber 540 and the dialysis sorbent 520 (via the first fluid flow path in an outflow direction).

With reference to FIG. 5A (toxin clearance mode), during the outflow stage, the fluid flows from the subject's 550 peritoneum along the first fluid flow path 590 through the first pump 510, the 3-way valve 560, the dialysis sorbent 520, optionally the mixer 570, and to the storage chamber 540. With reference to FIG. 5B, during the inflow stage, the fluid flows from the storage chamber 540 along the second fluid flow path through optionally the mixer 570, the 3-way valve 560, the first pump 510 and to the subject's 550 peritoneum. The sugar concentrate supply pump 530 may supply a sugar concentrate to the fluid flowing along the first fluid flow path in one or both of the outflow and inflow phases.

With reference to FIG. 5C, during the outflow stage in ‘UF only’ mode, the fluid flows from the subject's 550 peritoneum along the second fluid flow path through the first pump 510, the 3-way valve 560, optionally the mixer 570 and to the storage chamber 540. With reference to FIG. 5B, during the inflow stage in ‘UF only’ mode, the fluid flows from the storage chamber 540 along the second fluid flow path through optionally the mixer 570, the 3-way valve 560, the first pump 510 and to the subject's 550 peritoneum. The sugar concentrate supply pump 530 may supply a sugar concentrate to the fluid flowing along the second fluid flow path in either the inflow or outflow phase, or during both phases.

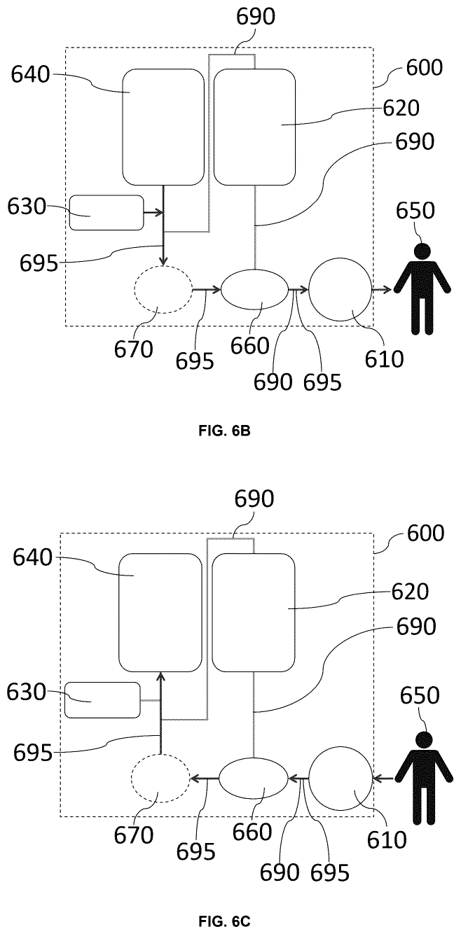

FIG. 6 depicts an apparatus 600 and a subject 650. The apparatus 600 includes a first pump 610, a dialysis sorbent 620, a sugar concentrate supply pump 630, a storage chamber 640, a 3-way valve 660, optionally a mixer 670 supplied to the fluid at a location between the sugar concentrate supply pump 630 and the 3-way valve 660, a first fluid flow path 690 and a second fluid flow path 695. The sugar concentrate supply pump 630 is supplied to the fluid at a location between the storage chamber 640 and the 3-way valve 660 (in the second fluid flow path in an inflow or outflow direction) or between the storage chamber 640 and the dialysis sorbent 620 (via the first fluid flow path in an outflow direction). In the apparatus 600, there is also a first fluid flow path from the first pump 610, through the 3-way valve 660, the dialysis sorbent 620 and to the storage chamber 640, and a second fluid flow path from the first pump 610, through the 3-way valve 660, optionally the mixer 670, and to the storage chamber 640.

With reference to FIG. 6A (toxin removal mode), during the outflow stage, the fluid flows from the subject's 650 peritoneum along the first fluid flow path through the first pump 610, the 3-way valve 660, the dialysis sorbent 620 and to the storage chamber 640, and the fluid also flows from the subject's 650 peritoneum along the second fluid flow path through the first pump 610, the 3-way valve 660, optionally the mixer 670 and to the storage chamber 640. The sugar concentrate supply pump 630 may supply a sugar concentrate to the fluid flowing along the first fluid flow path. With reference to FIG. 6B, during the inflow phase, the fluid flows from the storage chamber 640 along the second fluid flow path through optionally the mixer 670, the 3-way valve 660, the first pump 610 and to the subject's 650 peritoneum. The sugar concentrate supply pump 630 may supply a sugar concentrate to the fluid flowing along the second fluid flow path.

With reference to FIG. 6C, during the outflow stage in ‘UF only’ mode, the fluid flows from the subject's 650 peritoneum along the second fluid flow path through the first pump 610, the 3-way valve 660, optionally the mixer 670 and to the storage chamber 640. With reference to FIG. 6B, during the inflow stage in ‘UF only’ mode, the fluid flows from the storage chamber 640 along the second fluid flow path through optionally the mixer 670, the 3-way valve 660, the first pump 610 and to the subject's 650 peritoneum. The sugar concentrate supply pump 630 may supply a sugar concentrate to the fluid flowing along the second fluid flow path in either the inflow and outflow phases.

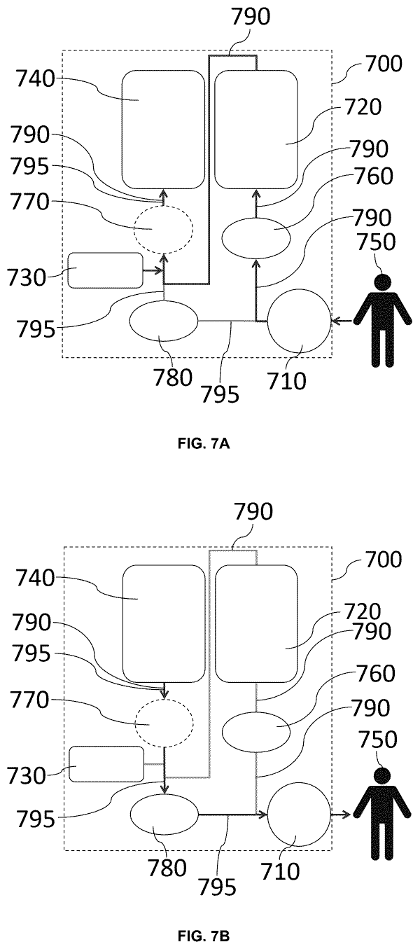

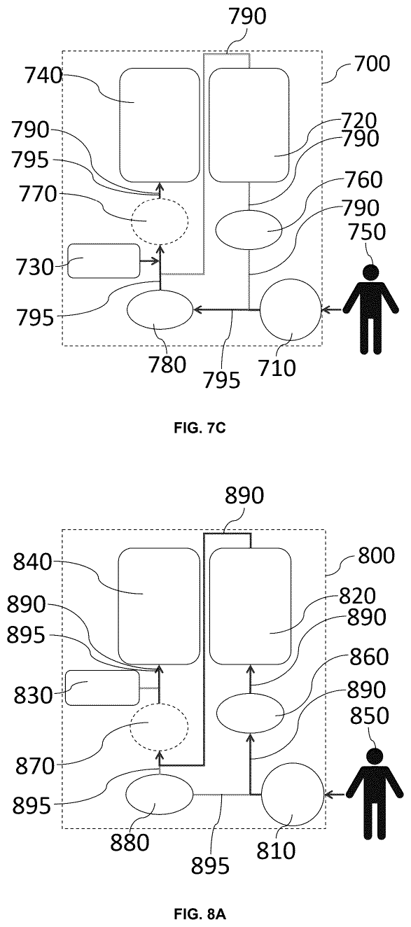

FIG. 7 depicts an apparatus 700 and a subject 750. The apparatus 700 includes a first pump 710, a dialysis sorbent 720, a sugar concentrate supply pump 730, a storage chamber 740, a first 2-way valve 760, a second 2-way valve 780, optionally a mixer 770 supplied to the fluid at a location between the sugar concentrate supply pump 730 and the storage chamber 740, a first fluid flow path 790 and a second fluid flow path 795.

The sugar concentrate supply pump 730 is supplied to the fluid at a location between the storage chamber 740 and the second 2-way valve 780 and between the storage chamber 740 and the dialysis sorbent 720. In the apparatus 700, there is also a first fluid flow path from the first pump 710, through the first 2-way valve 760, the dialysis sorbent 720, optionally the mixer 770 and to the storage chamber 740, and a second fluid flow path from the first pump 710, through the second 2-way valve 780, optionally the mixer 770, and to the storage chamber 740.

With reference to FIG. 7A (toxin removal mode), during the outflow stage, the fluid flows from the subject's 750 peritoneum along the first fluid flow path through the first pump 710, the first 2-way valve 760, the dialysis sorbent 720, optionally the mixer 770 and to the storage chamber 740. The sugar concentrate supply pump 730 may supply a sugar concentrate to the fluid flowing along the first fluid flow path. With reference to FIG. 7B, during the inflow stage, the fluid flows from the storage chamber 740 along the second fluid flow path through optionally the mixer 770, the second 2-way valve 780, the first pump 710 and to the subject's 750 peritoneum. The sugar concentrate supply pump 730 may supply a sugar concentrate to the fluid flowing along the second fluid flow path.

With reference to FIG. 7C, during the outflow stage in ‘UF only’ mode, the fluid flows from the subject's 750 peritoneum along the second fluid flow path through the first pump 710, the second 2-way valve 780, optionally the mixer 770 and to the storage chamber 740. The sugar concentrate supply pump 730 may supply a sugar concentrate to the fluid flowing along the second fluid flow path. With reference to FIG. 7B, during the inflow stage in ‘UF only’ mode, the fluid flows from the storage chamber 740 along the second fluid flow path through optionally the mixer 770, the second 2-way valve 780, the first pump 710 and to the subject's 750 peritoneum. The sugar concentrate supply pump 730 may supply a sugar concentrate to the fluid flowing along the second fluid flow path.

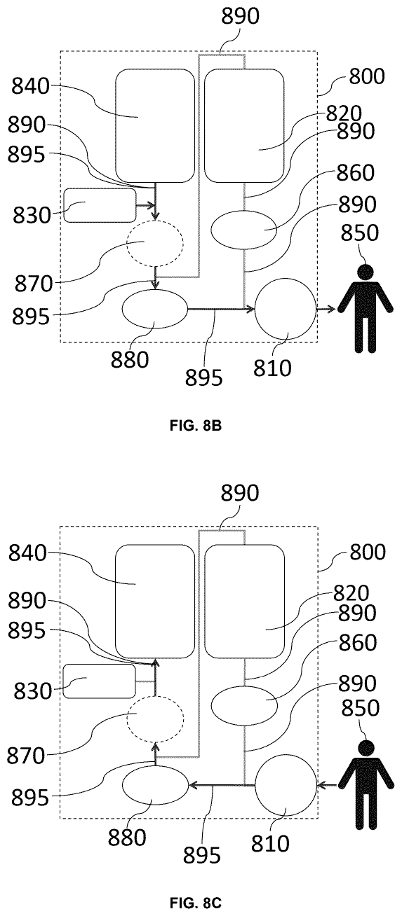

FIG. 8 depicts an apparatus 800 and a subject 850. The apparatus 800 includes a first pump 810, a dialysis sorbent 820, a sugar concentrate supply pump 830, a storage chamber 840, a first 2-way valve 860, a second 2-way valve 880, optionally a mixer 870 supplied to the fluid at a location between the sugar concentrate supply pump 830 and the second 2-way valve 880, a first fluid flow path 890 and a second fluid flow path 895. The sugar concentrate supply pump 830 is supplied to the fluid at a location between the storage chamber 840 and the second 2-way valve 880 in the second fluid flow path and between the storage chamber 840 and the dialysis sorbent 820 in the first fluid flow path. In the apparatus 800, there is also a first fluid flow path from the first pump 810, through the first 2-way valve 860, the dialysis sorbent 820, optionally the mixer 870 and to the storage chamber 840, and a second fluid flow path from the first pump 810, through the second 2-way valve 880, optionally the mixer 870, and to the storage chamber 840.

With reference to FIG. 8A (toxin clearance mode), during the outflow stage, the fluid flows from the subject's 850 peritoneum along the first fluid flow path through the first pump 810, the first 2-way valve 860, the dialysis sorbent 820, optionally the mixer 870 and to the storage chamber 840. The sugar concentrate supply pump 830 may supply a sugar concentrate to the fluid flowing along the first fluid flow path. With reference to FIG. 8B, during the inflow stage, the fluid flows from the storage chamber 840 along the second fluid flow path through optionally the mixer 870, the second 2-way valve 880, the first pump 810 and to the subject's 850 peritoneum. The sugar concentrate supply pump 830 may supply a sugar concentrate to the fluid flowing along the second fluid flow path.

With reference to FIG. 8C, during the outflow stage in ‘UF only’ mode, the fluid flows from the subject's 850 peritoneum along the second fluid flow path through the first pump 810, the second 2-way valve 880, optionally the mixer 870 and to the storage chamber 840. The sugar concentrate supply pump 830 may supply a sugar concentrate to the fluid flowing along the second fluid flow path. With reference to FIG. 8B, during the inflow stage in ‘UF only’ mode, the fluid flows from the storage chamber 840 along the second fluid flow path through optionally the mixer 870, the second 2-way valve 880, the first pump 810 and to the subject's 850 peritoneum. The sugar concentrate supply pump 830 may supply a sugar concentrate to the fluid flowing along the second fluid flow path.

FIG. 9 depicts an apparatus 900 and a subject 950. The apparatus 900 includes a first pump 910, a storage chamber 940, a sugar concentrate supply pump 930, and optionally a mixer 920 supplied to the fluid at a location between the sugar concentrate supply pump 930, the storage chamber 940 and a first fluid path 990. The sugar concentrate supply pump 930 is supplied to the fluid at a location between the storage chamber 940 and the first pump 910. In the apparatus 900, there is a fluid flow path from the first pump 910, optionally through the mixer 920 and to the storage chamber 940.

With reference to FIG. 9A, during the outflow stage, the fluid flows from the subject's 950 peritoneum along the fluid flow path through the first pump 910, optionally the mixer 920 and to the storage chamber 940. The sugar concentrate supply pump 930 supplies a sugar concentrate to the fluid flowing along the fluid flow path. With reference to FIG. 9B, during the inflow stage, the fluid flows from the storage chamber 940 along the fluid flow path through optionally the mixer 920, the first pump 910 and to the subject's 950 peritoneum.

As will be appreciated, this apparatus may be particularly useful for UF removal in heart patients who do not require toxin clearance, as it does not include a dialysis sorbent, thereby reducing the overall cost of the system in question. Water removal can help to improve blood pressure and maintain normotension.

FIG. 10 depicts an apparatus 1000 and a subject 1050. The apparatus 1000 includes a first pump 1010, a storage chamber 1040, a sugar concentrate supply pump 1030, and optionally a mixer 1020 supplied to the fluid at a location between the sugar concentrate supply pump 1030 and the storage chamber 1040. The sugar concentrate supply pump 1030 is supplied to the fluid at a location between the storage chamber 1040 and the first pump 1010. In the apparatus 1000, there is a fluid flow path 1090 from the first pump 1010, optionally through the mixer 1020 and to the storage chamber 1040.

With reference to FIG. 10A, during the outflow stage, the fluid flows from the subject's 1050 peritoneum along the fluid flow path 1090 through the first pump 1010, optionally the mixer 1020 and to the storage chamber 1040. With reference to FIG. 10B, during the inflow stage, the fluid flows from the storage chamber 1040 along the fluid flow path 1090 through optionally the mixer 1020, the first pump 1010 and to the subject's 1050 peritoneum. The sugar concentrate supply pump 1030 supplies a sugar concentrate to the fluid flowing along the fluid flow path.

As will be appreciated, this apparatus may also be particularly useful for UF removal in heart patients who do not require toxin clearance.

In the methods and apparatuses disclosed herein, it may be desired to remove fluid from the subject through their use. This may be because the subject cannot adequately remove fluid by conventional means or simply to prevent increased intraperitoneal volume due to accumulation of ultrafiltration. This may be achieved by removing a certain amount of ultrafiltration periodically from the subject. This can be done in either outflow or inflow. If done during outflow, the system may remove slightly more than the intended tidal volume (e.g. 270 ml for a 250 ml tidal volume). The additional volume may be directed towards an Ultrafiltration Bag that is connected to the apparatus. This can be controlled by the controller and the Ultrafiltration Bag may be connected in any suitable position to receive this excess volume of the second hypertonic solution. If ultrafiltration removal is done during inflow, a portion of the tidal volume is not returned to the patient but is redirected to the attached Ultrafiltration Bag instead (e.g. 230 ml is put back to a patient for a 250 ml tidal volume). Again, this can be controlled by the controller and the Ultrafiltration Bag may be connected in any suitable position to receive this excess volume of the third hypertonic solution. This step of ultrafiltration removal is not done every cycle but may be calculated to be removed periodically, depending on the expected ultrafiltration that is to be generated by the subject in question. This step may also be omitted completely if the subject is expected to remove minimal ultrafiltration or if the patient is underfilled with the intention of retaining the ultrafiltration within the peritoneum itself without causing increased intraperitoneal volume.

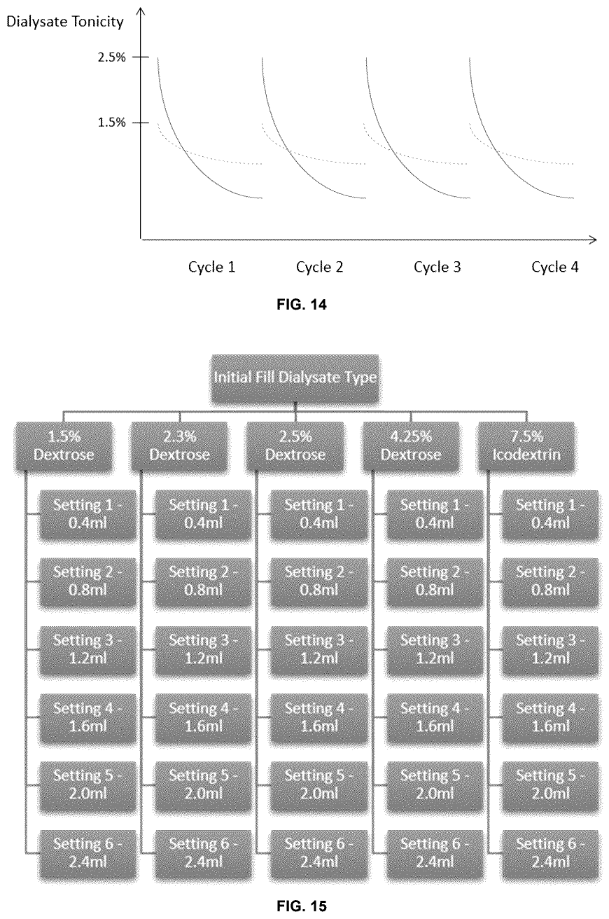

The apparatus and methods used herein can also be applied to regular APD modalities to:
a) Obtain more UF without resorting to higher Fill tonicity. For example, a patient that require 2.5 wt % Dextrose Fills can potentially apply the disclosed apparatus to start with 1.5 wt % Dextrose Fills and supplement it with additional glucose throughout each dwell to obtain higher UF efficiency. FIG. 14 shows the potential dialysate tonicity for each dwell (standard 2.5 wt % Dextrose APD Fills in solid lines and AGMS with 1.5 wt % Fills in dotted lines). This can reduce the total amount of glucose exposure and protect the peritoneal membrane compared to standard APD.
b) Remove the requirement for a day dwell (especially for high transporters) in patients who require more UF that can be typically achieved with a standard overnight APD therapy. With higher UF efficiency, more UF can be drawn from the patient within the same period (overnight therapy) which can be beneficial in terms of:
   a. The patient does not require an additional bag of dialysate for day dwell. This saves on needing to purchase and stock large quantities of dialysate.
   b. Patients who are allergic to Icodextrin but require a day dwell. In order to achieve sufficient UF with these patient types and to ensure no UF reabsorption occurs, the day dwell typically requires a higher tonicity dialysate (e.g. 4.25 wt % Dextrose). In this case, patients are exposed to even higher amount of glucose which can accelerate ultrafiltration failure. Application of the AGMS can remove the need for high tonicity Fills for the day dwell and prolong peritoneal membrane function.

AGMS can be achieved without a dialysate sorbent being installed in the apparatus. For example, the Tidal Volume can be removed from the patient and glucose can be diluted/mixed into the Tidal Volume before being introduced back to the patient.

Further aspects and embodiments of the invention will now be described by reference to the following non-limiting examples.

### EXAMPLES

### Materials

The functional model to provide necessary pumps/valves/storage module was purchased from: valves: Clippard Valves-Clippard NPV-3-1C-25-12; Main Pump-Boxer 24V stepper 25057.000; Glucose Pump-Prosense NE500; Pressure Sensors-Omega PX409-030AUSBH; Electronics-Arduino Due and relevant supporting electronics. 70% glucose concentrate solution was Glucose Injection (Baxter), 70% solution for infusion AHB0293.

The dialysis sorbent used can be any suitable material that can be packed into the system. For example, the sorbent may be any of those disclosed in PCT Application No. PCT/SG2009/000229. In particular, the sorbent was prepared according to example 1g-1 on p36 at [13].

### Example 1. In Vivo AWAK AGMS Tidal Therapy

The AWAK peritoneal dialysis system uses a tidal therapy that differs from the regular automated peritoneal dialysis (APD)/Continuous ambulatory peritoneal dialysis (CAPD) modalities by continuously regenerating a small volume of dialysate (relative to the Initial Fill volume). This is known as the Tidal Volume. At each cycle, the AWAK peritoneal dialysis system introduces a small volume of glucose to ‘top up’ and counter any loss of osmotic pressure due to the dilution effect of UF generated as well as any lymphatic absorption of glucose by the patient. The aim of this system is to maintain a steady and mildly hypertonic solution which allows consistent UF generation throughout the entire therapy.

### Animal Studies

Several animal studies were conducted with a functional model that allowed a similar glucose dosing as the AWAK AGMS. The study was conducted on several different 5/6 nephrectomised pigs at different settings and compared to a standard 10-hour APD therapy. An Initial Fill of 1.5% Dextrose dialysate was used for the AGMS study and glucose dosing was done once every 7.5 mins for a total therapy duration of 7 hours. The Tidal Volume for the AGMS was 250 mL. UF was calculated according to the formula below:

Ultrafiltration=Final Drain−Initial Fill−Additional Glucose Dosing Volume

The APD reference was done with 5× Fills of 1.5% Dextrose dialysate for a total therapy duration of 10 hours.

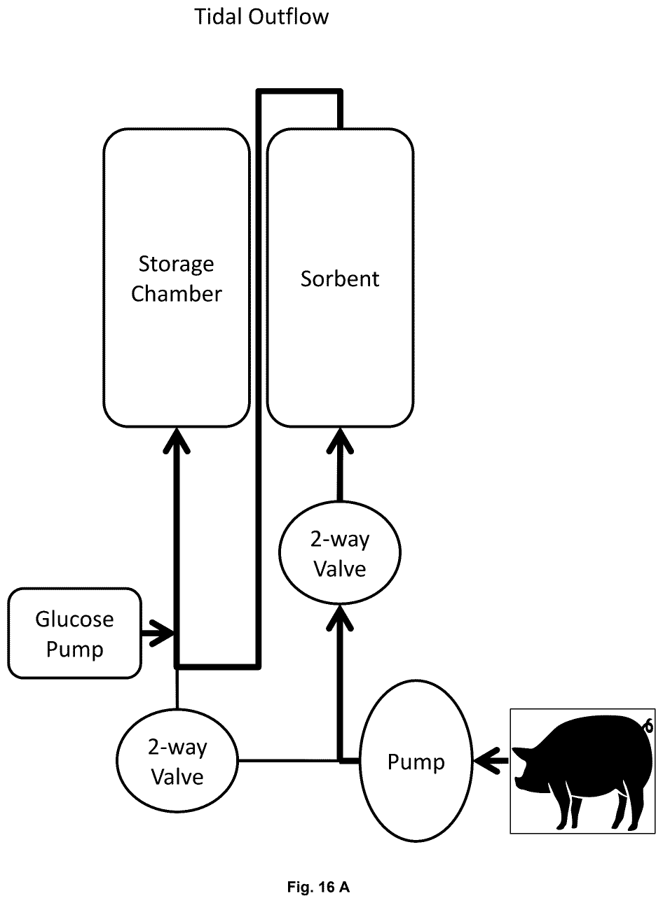

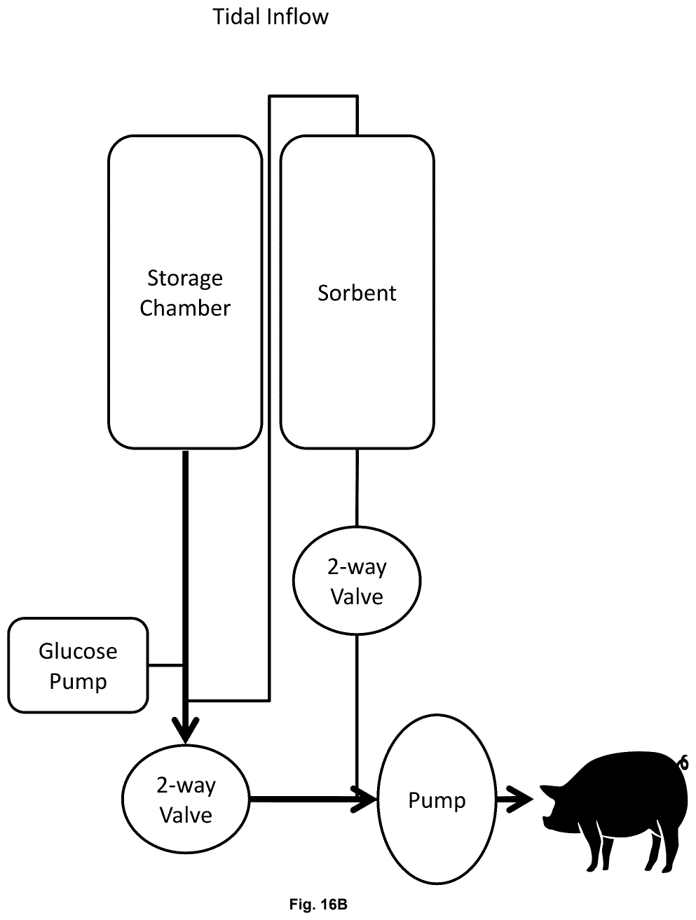

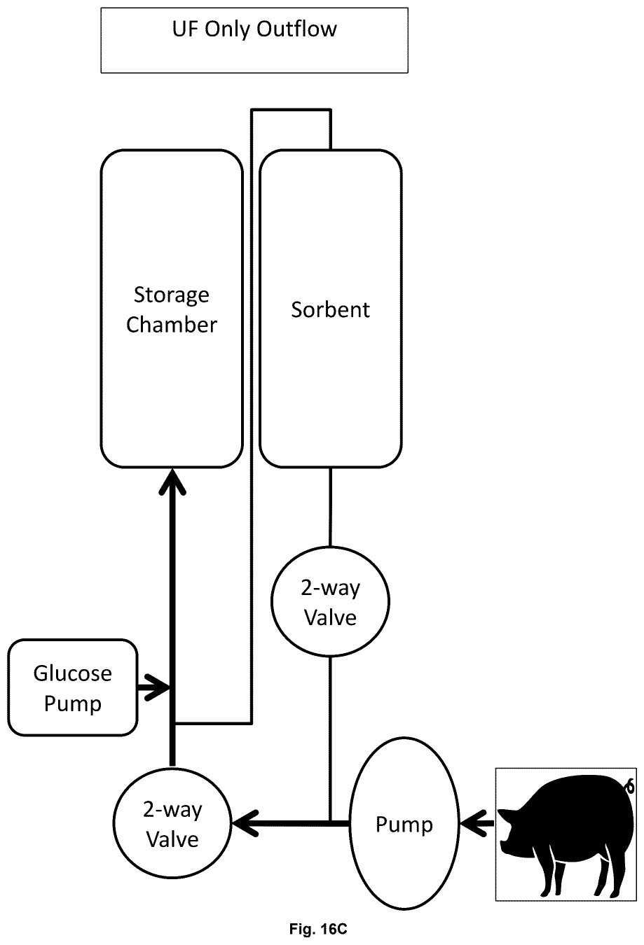

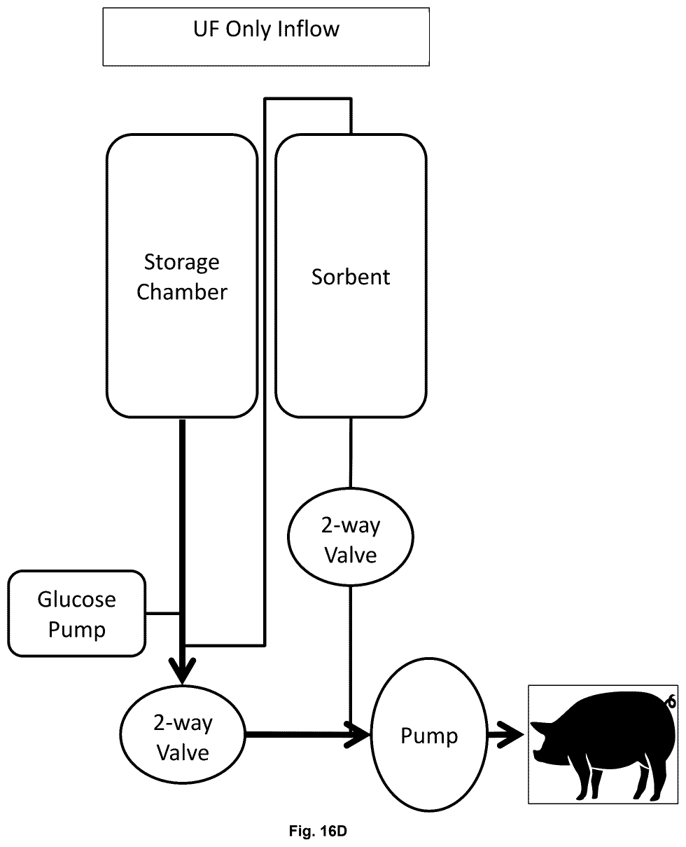

The operation of the apparatus used for this method is illustrated in FIGS. 16A to 16D. FIG. 16A depicts the tidal outflow flow path from a pig through the dialysis sorbent to the storage chamber, where glucose is introduced into the outflow fluid as it flows to the storage chamber. FIG. 16B depicts the inflow flow path back to the pig, where no glucose is added to the fluid leaving the storage chamber. FIG. 16C depicts an ultrafiltration only outflow cycle, where the fluid from the pig goes directly to the storage chamber with glucose addition, but does not pass through the dialysis sorbent. FIG. 16D depicts the inflow path and is essentially identical to FIG. 16B in operation.

### Calculation of UF Efficiency

To determine if AWAK AGMS is more efficient at producing UF, the UF efficiency per gram of glucose exposure was calculated according to the formula below:

$$Ultrafiltration Efficiency per gram of Glucose Exposure = \frac{Ultrafiltration ( ml )}{Total Glucose Exposure ( g )}$$

UF value was obtained from the device display.

### Results and Discussion

The UF obtained for one set of experiments is shown in Table 1.

TABLE 1
UF obtained from a study on a 5/6 nephrectomised pig.

| | 7 h Setting 1 | 7 h Setting 2 | 7 h Setting 3 | 10 h APD |
|---|---|---|---|---|
| | UF | UF | UF | UF |
| | (n = 7) | (n = 7) | (n = 16) | (n = 6) |
| P2551 | 298 ml | 498 ml | 678 ml | 822 ml |
| Ultra- | | | | |
| filtration | | | | |

The corresponding glucose (monohydrate) exposure is shown in Table 2. Glucose exposure is defined as the total amount of glucose that is presented to the patient (to the pig in this case) throughout the entire therapy. In the case of AWAK AGMS, this includes the glucose that is present in the Initial Fill.

TABLE 2
Total glucose exposure on the pig.

| | 7 h Setting 1 | 7 h Setting 2 | 7 h Setting 3 | 10 h APD |
|---|---|---|---|---|
| | Glucose | Glucose | Glucose | Glucose |
| | Exposure | Exposure | Exposure | Exposure |
| P2551 | 46 g | 61 g | 77 g | 150 g |
| Ultra- | | | | |
| filtration | | | | |

Lower total glucose exposure is considered beneficial in peritoneal dialysis as hypertonic solutions are a known cause for peritoneal membrane changes, which can ultimately lead to UF failure. Once a peritoneal dialysis patient reaches UF failure, they can no longer continue on peritoneal dialysis and will need to switch to another treatment modality. Therefore, any peritoneal dialysis treatment modality that can reduce glucose exposure to the patient can potentially extend the time that the patient can remain on peritoneal dialysis.

A higher UF efficiency would result in lower glucose exposure per therapy. With lower glucose exposure, it is expected that the patient will experience less peritoneal membrane changes over time and can potentially stay on peritoneal dialysis for longer. As shown in Table 3, UF efficiency for AWAK AGMS increases at higher glucose setting and is more efficient compared to a standard APD therapy.

TABLE 3
UF efficiency per gram of glucose exposure.

| | 7 h Setting 1 | 7 h Setting 2 | 7 h Setting 3 | 10 h APD |
|---|---|---|---|---|
| | UF per | UF per | UF per | UF per |
| | Gram | Gram | Gram | Gram |
| | Glucose | Glucose | Glucose | Glucose |
| | Exposure | Exposure | Exposure | Exposure |
| P2551 | 6.53 ml/g | 8.12 ml/g | 8.81 ml/g | 5.48 ml/g |
| Ultra- | | | | |
| filtration | | | | |

This means that with the AGMS, the patient is able to:
a) generate the same amount of UF with less glucose exposure;
b) generate more UF for the same glucose exposure; or
c) generate more UF for a similar therapy duration.

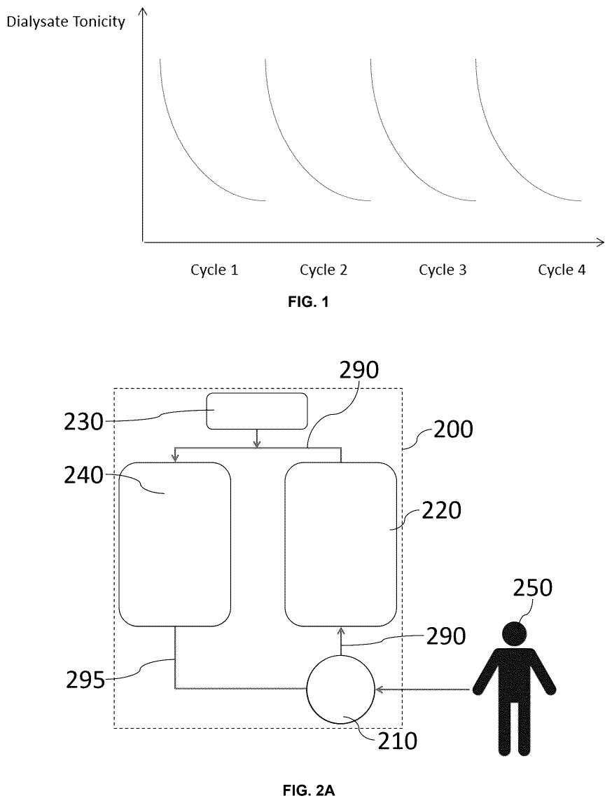

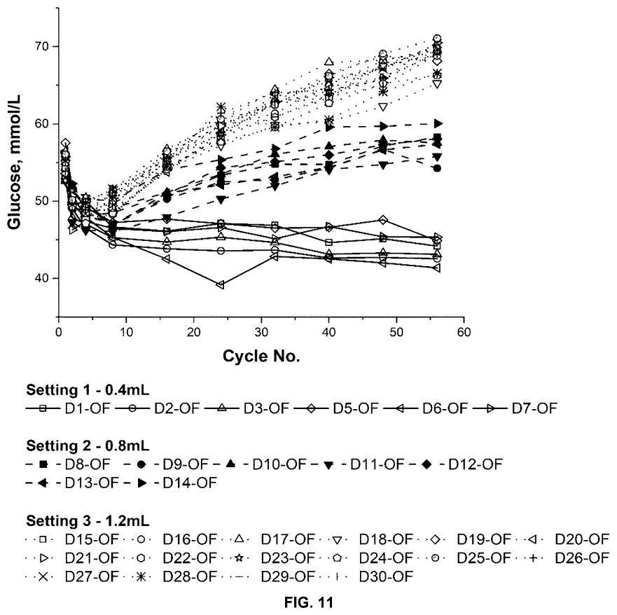

Another benefit of the AWAK AGMS tidal therapy is the concentration gradient of the glucose. The AWAK AGMS tidal therapy will only require one hypertonic Initial Fill. The device subsequently tries to maintain a sustained UF generated by maintaining a mildly hypertonic solution within the patient's peritoneum. FIG. 11 shows the tonicity of the dialysate that was drawn from the patient in outflow. This tonicity would indicate the concentration that exists in the patient's peritoneum. An Initial Fill of 1.5% Dextrose (˜76 mmol/L) was used. With a sufficiently high setting, the mildly hypertonic solution may end up being of similar hypertonicity as the Initial Fill volume. This contrasts with the standard APD therapy that introduces multiple instances of hypertonic Fills (FIG. 1).

### Example 2. Adequate UF by Personalizing the Glucose Dosing

In sorbent based peritoneal dialysis, spent dialysate is processed continuously and the regenerated dialysate is reconstituted with glucose, calcium and magnesium before returning to the patient's peritoneum. Continuous glucose infusion allows steady and sustained UF production. UF requirements vary from patient to patient and in standard-of-care (SOC) peritoneal dialysis modalities, targeted daily UF volume can be regulated by controlling the concentration of dialysis solutions used. However, in order to maintain an osmotic gradient for the targeted UF, a higher initial glucose concentration is required. Long term exposure to higher glucose concentrations may eventually lead to loss of peritoneal membrane function and ultimately cause UF failure. The aim of the study was to determine if adequate UF can be met by personalizing the glucose dosing during sorbent based therapy according to the patient's UF requirement.

### Personalization of Glucose Dosing

5/6 nephrectomised pig (Sus Scrofa species, male, weighing 90-100 kg) were used. In the pre-study, continuous cycling of peritoneal dialysis was carried out for 14 weeks. The study duration were 6 days (control) and 30 days (treatment). In the control (6 days) group, the APD consists of 10 hours of therapy daily with 2 L, Low Cal Dianeal® with 1.5% dextrose. In the treatment (30 days) group, the sorbent-based peritoneal dialysis consists of 7 hours of tidal therapy daily with 2 L, Low Cal Dianeal® with 1.5% dextrose.

After the initial fill, regenerated dialysate was re-infused with various glucose settings. The three different glucose settings S1, S2 and S3 used had a total glucose exposure ranging from 15.7 g to 47.0 g. UF volume, glucose absorption and exposure were recorded and analysed.

### Results and Discussion

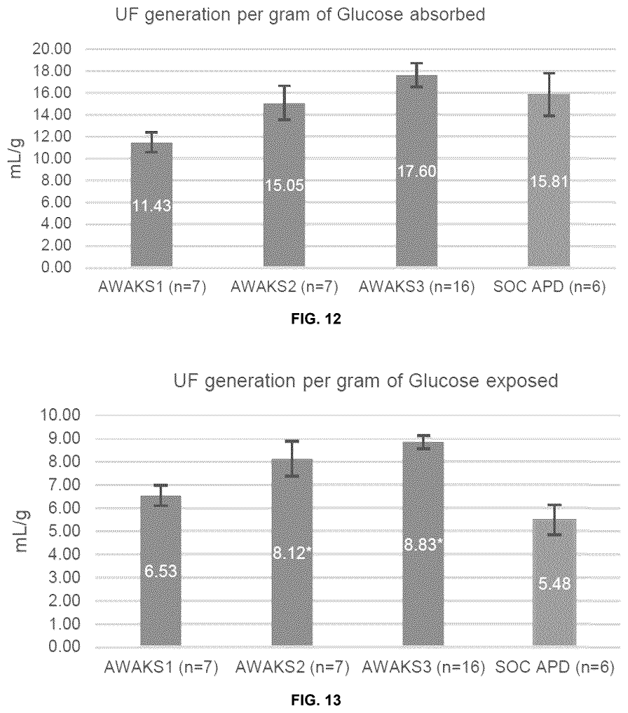

The animal underwent 6 days of conventional APD therapy followed by 30 days of sorbent-based peritoneal dialysis therapy with 3 different glucose settings. More than 2-fold increase in UF was observed when Glucose Setting 1 (298, SD=54 mL) was changed to Setting 3 (680, SD=85 mL, p<0.001) in the same animal. No significant differences were found in UF generated per gram of glucose monohydrate absorbed in all AWAK settings compared to SOC (FIG. 12). UF generated per gram of glucose monohydrate exposure (FIG. 13) for Setting 2 8.12, SD=2.0 mL/g) and Setting 3 (8.83, SD=1.1 mL/g) were significantly higher when compared to conventional APD therapy (5.48, SD=1.6 mL/g; p<0.05).

This study has demonstrated that UF can be augmented with alteration of glucose settings in AWAK's sorbent-based tidal peritoneal dialysis therapy. Although UF per gram of glucose absorbed is not different from SOC, UF per gram of glucose exposed significantly favors AWAK's AGMS. This can be explained by the tidal peritoneal dialysis mode where the continuous infusion of regenerated dialysate allows AWAK to provide a sustained osmotic gradient for UF generation.

What is claimed:

1. A method of removing fluid from a subject, comprising the steps of:
(i) administering from 200 mL to a tolerable maximum volume for the subject of a first hypertonic solution comprising a sugar-based osmotic agent to a peritoneal cavity in the subject;
(ii) allowing water from the subject to pass into the peritoneal cavity by osmosis, thereby forming a second hypertonic solution within the peritoneal cavity, the second hypertonic solution having a lower sugar concentration than the first hypertonic solution;
(iii) withdrawing from 100 to 500 mL of the second hypertonic solution from the peritoneal cavity and combining the withdrawn second hypertonic solution with from 0.3 to 6.8 mL of a sugar concentrate to form a third hypertonic solution, the sugar concentrate having a concentration of from 0.25 to 0.9 g/mL;
(iv) administering the third hypertonic solution to the peritoneal cavity to form a fourth hypertonic solution within the peritoneal cavity by mixing of the second and third hypertonic solutions; and
(v) repeating steps (ii) to (iv) every 5 to 30 minutes for a desired treatment time.

2. The method according to claim 1, wherein step (i) comprises administering from 300 to 4,000 mL of a first hypertonic solution comprising a sugar-based osmotic agent to the peritoneal cavity.

3. The method according to claim 1, wherein step (i) comprises administering from 1,000 to 2,500 mL of a first hypertonic solution comprising a sugar-based osmotic agent to the peritoneal cavity.

4. The method according to claim 1, wherein step (iii) comprises withdrawing from 200 to 400 mL of the second hypertonic solution.

5. The method according to claim 1, wherein step (iii) comprises combining the withdrawn second hypertonic solution with from 0.3 to 6.8 mL of a sugar concentrate.

6. The method according to claim 5, wherein step (iii) comprises combining the withdrawn second hypertonic solution with from 0.5 to 2.4 mL of a sugar concentrate.

7. The method according to claim 1, wherein step (iii) further comprises the periodical step of removing fluid from the subject by:
(a) withdrawing a volume of the second hypertonic solution that comprises a desired second hypertonic volume and an excess second hypertonic volume, and removing the excess second hypertonic volume before forming the third hypertonic solution; or
(b) withdrawing a volume of the second hypertonic solution that comprises a desired second hypertonic volume and an excess second hypertonic volume, then forming a third hypertonic solution that comprises a desired third hypertonic volume and an excess third hypertonic volume and removing the excess third hypertonic volume before administering the third hypertonic solution to the subject.

8. (canceled)

9. The method according to claim 1, wherein the desired treatment time is from 7 to 10 hours.

10. The method according to claim 1, wherein step (iii) comprises passing the withdrawn second hypertonic solution through a dialysis sorbent; or passing the third hypertonic solution through a dialysis sorbent.

11. The method according to claim 10, further comprising an initial step of saturating the sorbent with the sugar concentrate.

12. The method according to claim 1, wherein the sugar concentrate comprises a sugar-based osmotic agent.

13. The method according to claim 1, wherein the sugar-based osmotic agent concentration of the withdrawn second hypertonic solution in each repetition of step (ii) varies by less than 50% relative to the initial sugar-based osmotic agent concentration of the withdrawn second hypertonic solution the first time step (ii) is performed

14. The method according to claim 1, wherein the third hypertonic solution has a higher sugar-based osmotic agent concentration than the first hypertonic solution.

15. The method according to claim 1, wherein the fourth hypertonic solution has a sugar-based osmotic agent concentration that is less than or equal to the sugar-based osmotic agent concentration of the first hypertonic solution.

16. An apparatus comprising:
a first pump fluidly connectable to a subject's peritoneal cavity;
a sugar concentrate supply pump connectable to a source of sugar concentrate;
a storage chamber; and
a first fluid flow path from the first pump to the storage chamber;
where the first pump is configured to pump fluid in either direction along the first fluid flow paths,
such that when in use:
fluid can be drawn from a subject's peritoneal cavity along the first fluid flow path by the first pump to the storage chamber,
and
the sugar concentrate supply pump is configured to supply a sugar concentrate to the fluid flowing along the first fluid flow path.

17. (canceled)

18. The apparatus according claim 16, further comprising one or more mixers located on the first fluid flow path.

19. An apparatus comprising:
a first pump fluidly connectable to a subject's peritoneal cavity;
a sugar concentrate supply pump connectable to a source of sugar concentrate;
a storage chamber;
a first fluid flow path from the first pump to the storage chamber;
a dialysis sorbent situated in the first fluid flow path; and
a second fluid flow path from the first pump to the storage chamber that bypasses the sorbent, wherein the apparatus is configured to be connected to a controller configured to operate the apparatus, where the controller can select whether to use the first fluid flow path or the second fluid flow path for any fluid flow operation, where the first pump is configured to Jump fluid in either direction along the first fluid flow path or second fluid flow path, such that when in use:
fluid can be drawn from a subject's peritoneal cavity along the first fluid flow path or second flow fluid path by the first pump to the storage chamber; and
the sugar concentrate supply pump is configured to supply a sugar concentrate to fluid flowing along the first fluid flow path or second fluid flow path.

20. The apparatus according to claim 19, further comprising one or more valves configured to selectively enable fluid flow through one of the first and second fluid flow paths.

21. The apparatus according to claim 19, further comprising one or more mixers located on the first and second fluid flow paths.

22. (canceled)

23. The apparatus according to claim 19, wherein the dialysis sorbent is situated upstream or downstream of the storage chamber in the first fluid flow path during the outflow phase, or the dialysis sorbent is situated downstream of the storage chamber in the fluid flow path during the inflow phase.
